(19) United States
(12) Patent Application Publication
(10) Pub. No.: US 2020/0111546 A1
(43) Pub. Date: Apr. 9, 2020

(54) Automatic Detection and Reporting of Medical Episodes in Patient Medical History

(71) Applicant: International Business Machines Corporation, Armonk, NY (US)

(72) Inventors: Tanveer F. Syeda-Mahmood, Cupertino, CA (US); Marina Bendersky, Cupertino, CA (US)

(21) Appl. No.: 16/151,429

(22) Filed: Oct. 4, 2018

(51) Int. Cl. G16H 10/60; G16H 15/00; G16H 30/00

(52) U.S. Cl. CPC G16H 10/60; G06F 21/6245; G16H 30/00; G16H 15/00

(57) ABSTRACT

A medical episode analysis mechanism is provided. The mechanism analyzes patient information to extract a plurality of medical concept instances corresponding to medical characteristics associated with a patient. The mechanism generates a multi-dimensional time series data structure representing a clinical history timeline of the medical concept instances. The mechanism analyzes the multi-dimensional time series data structure to identify one or more medical episodes in the clinical history timeline, where a medical episode is a group of related encounters between one or more medical services providers and the patient. The mechanism outputs a visual representation of the identified one or more medical episodes to a computing device, where the visual representation provides a navigation user interface through which a user may browse the clinical history timeline associated with the patient.

### BACKGROUND

The present application relates generally to an improved data processing apparatus and method and more specifically to mechanisms for performing automatic detection and reporting of medical episodes in patient medical history.

Information technology trends in the practice of medicine have made a push to integrate medical records of various computing systems, such as in consolidated patient electronic medical record (EMR) repositories. As such, medical personnel have access to larger sets of medical data for their patients. However, this leads to problems in that the medical personnel must sift through the large amounts of available data to find the data that is of interest to them and their current purpose for accessing the data, i.e. typically for the purpose of treating the patient.

### SUMMARY

This Summary is provided to introduce a selection of concepts in a simplified form that are further described herein in the Detailed Description. This Summary is not intended to identify key factors or essential features of the claimed subject matter, nor is it intended to be used to limit the scope of the claimed subject matter.

In one illustrative embodiment, a method is provided, in a data processing system comprising a processor and a memory, the memory comprising instructions executed by the processor to configure the processor to implement a medical episode analysis engine. The method comprises analyzing, by the medical episode analysis engine, patient information to extract a plurality of medical concept instances corresponding to medical characteristics associated with a patient. The method further comprises generating, by the medical episode analysis engine, a multi-dimensional time series data structure representing a clinical history timeline of the medical concept instances. Moreover, the method comprises analyzing, by the medical episode analysis engine, the multi-dimensional time series data structure to identify one or more medical episodes in the clinical history timeline. A medical episode is a group of related encounters between one or more medical services providers and the patient. Furthermore, the method comprises outputting, by the medical episode analysis engine, a visual representation of the identified one or more medical episodes to a computing device. The visual representation provides a navigation user interface through which a user may browse the clinical history timeline associated with the patient.

In other illustrative embodiments, a computer program product comprising a computer useable or readable medium having a computer readable program is provided. The computer readable program, when executed on a computing device, causes the computing device to perform various ones of, and combinations of, the operations outlined above with regard to the method illustrative embodiment.

In yet another illustrative embodiment, a system/apparatus is provided. The system/apparatus may comprise one or more processors and a memory coupled to the one or more processors. The memory may comprise instructions which, when executed by the one or more processors, cause the one or more processors to perform various ones of, and combinations of, the operations outlined above with regard to the method illustrative embodiment.

These and other features and advantages of the present invention will be described in, or will become apparent to those of ordinary skill in the art in view of, the following detailed description of the example embodiments of the present invention.

### BRIEF DESCRIPTION OF THE DRAWINGS

The invention, as well as a preferred mode of use and further objectives and advantages thereof, will best be understood by reference to the following detailed description of illustrative embodiments when read in conjunction with the accompanying drawings, wherein:

FIG. 1 is an example of a portion of CERs that may be used to extract instances of CERs from patient information in a patient EMR which may constitute a problem list for the patient;

FIG. 2A illustrates the problem list that may be extracted from the patient medical information of the patient EMR along with timestamp information;

FIG. 2B shows a timeline plot in which spikes in the timeline plot are indicative of a medical episode;

FIG. 2C shows another lot of a problem list against time on a different time scale;

FIGS. 3A and 3B provide an example diagrams illustrating a combination of the CER instances along a common time line in accordance with one illustrative embodiment;

FIG. 4A is an example diagram illustrating a multi-dimensional CER time series combining multiple individual CER time series in accordance with one illustrative embodiment;

FIG. 4B is an example curve as a function of scale in accordance with one illustrative embodiment;

FIG. 4C is an example diagram illustrating inflection points and their corresponding rank in scale space in accordance with one illustrative embodiment;

FIG. 5 depicts a schematic diagram of one illustrative embodiment of a cognitive health computing system implementing a request processing pipeline in a computer network, in which medical episode identification and summarization is performed;

FIG. 6 is an example block diagram of a data processing system in which aspects of the illustrative embodiments may be implemented; and

FIG. 7 is a flowchart outlining an example operation for performing medical episode identification and summarization in accordance with one illustrative embodiment.

### DETAILED DESCRIPTION

As mentioned above, recent trends in medical information technology is to integrate computing systems from various sources, e.g., hospitals, clinics, doctor offices, medical insurance companies, pharmacies, medical laboratories, etc., such that a comprehensive patient electronic medical record (EMR) is generated for patients compiling all of their medical information from the various source computing systems. As such, a large amount of data is available to medical personnel to assist with evaluating the medical conditions, medical history, and the like, of the patient, all with an aim at improving the way in which the patient receives treatments for their medical condition. Of course, other uses for such consolidated patient EMR data may be made as well by various users depending on the desired implementations, e.g., statistical analysis, outbreak detection, etc.

Problems exist when such large amounts of data are compiled in that the volume of data often makes it difficult to pinpoint or identify the particular information about a patient that a medical professional is most interested in. For example, a typical patient's record over a period of 10 or more years could easily have 100,000 elements of information ranging from problem lists, recorded diagnosis, visit summaries, medications, labs, etc. One solution to this problem is to organize the patient data into medical episodes that the patient has experienced. A medical episode is a set of medical encounters with the patient, medical diagnoses, treatments, and the like, that are related to one another and may be associated with a same set of one or more medical conditions for which the patient has sought medical assistance.

Medical personnel may manually reason over a patient's clinical history based on documented encounters and follow-up visits all relating to a common originating problem description. These related documented elements are conceptually considered by the medical personnel to be medical episodes. Viewing the patient's medical records in terms of medical episodes is one way to summarize the information in a patient timeline and provides a birds-eye view of the patient's condition. Currently, such summaries are manually formed through such manual reasoning and are subject to errors if all related elements of the patient record are not easily found though manual examination or if non-related elements are mistakenly included in an episode.

Processing the compiled patient electronic medical record (EMR) comprising data from a variety of different source computing systems, may result in a “problem list” being generated from the EMR indicating the various medical conditions or problems that the patient has experienced or which are currently associated with the patient. While this problem listing may be obtained from the patient EMR, many times the listing does not provide a good sense of when events occurred and how often such events occurred with regard to these medical conditions. While the events may be ordered in time and sorted by date, there still may not be a sense of the medical episodes, e.g., groupings of patient encounters in a patient clinical timeline indicative of the fact that the encounters are related to one another. To the contrary, the problems in the problem list may appear to be separate instances without any relation to one another.

The illustrative embodiments provide a computerized tool for performing automatic detection and reporting of medical episodes in patient medical history. The detection of the medical episodes is based on the generation of time series of various comparative effective research (CER) elements present in the patient EMR data. A CER is a category of medical concepts, e.g., symptoms or medical condition signs, different types of clinical values or laboratory results, medications, diagnosis, medical conditions, etc. These CERs may be predefined. The CERs are time varying and are mapped to a time series for each CER (or medical concept category). Each type of CER, e.g., problem list, medications, etc., may have multiple CER elements which are instances of the CER, e.g., Bradycardia, Dyspnea, Gout, etc., are all instances or CER elements of a CER “problem list”. Because the CERs vary over time, and patients often encounter the same medical events over time, there may be multiple CER elements that repeatedly appear in the time series of the CER, such as based on repeated encounters and visits to medical personnel or facilities for reoccurring problems, or for the same problem, but with different medical personnel or facilities.

Each of the CERs may be plotted as a time series, recording their CER elements and applicable time period. The time series for the various CERs may be normalized such that a multi-dimensional cumulative CER time series is generated by the combination of time series using a normalized time scale. Scale space analysis, e.g., Gaussian scale space analysis, is applied to the multi-dimensional time series to identify major inflection points in the multi-dimensional time series. These inflection points identify the boundaries of medical episodes, with CER instances between the inflection points, and inclusive of the inflection end points, being the CER instances associated with the particular medical episode.

Once the medical episodes are identified through the above process and application of the mechanisms of the illustrative embodiments to the patient EMR, the individual episodes can be further grouped by analyzing the correlation between the events captured in the individual episodes using medical knowledge resources, ontologies, or logic that specifies medical episode events that are related to one another. For example, valvular disorders and atrial fibrillation reported in two different episodes could be grouped into a higher level summary group as ‘heart disorders’. If these co-occur with a prescription of diuretics, the higher level summary group may indicate some kidney disease as well. Thus, once the medical episodes are identified, the CERs of the individual medical episodes are analyzed to identify commonalities and correspondences between the CERs, which may be of different types, to identify a pattern of CERs indicative of a type of higher level medical episode, e.g., various medications being administered, symptoms, office visits to the doctor, diagnoses, etc., may all be correlated to identify a higher level medical episode.

Thus, the medical episode summaries may be generated across medical episodes by correlating the elements, e.g., CERs, of the medical episodes that are related to one another or constitute a pattern of CERs representative of a higher level medical episode. It should also be appreciated that such summaries may be generated across medical episodes that have overlapping or common CERs as well. That is, patterns of medical episodes may be identified which may then be correlated to a particular medical issue type which may then be used to summarize a plurality of medical episodes. For example, in some illustrative embodiments, groups of CERs that overlap may be generated in the manner described above, where the overlap is associated with common CERs associated with the different groups. For example, the groups of CERs defining medical episodes may be centered around a particular disease or other CERs and thus, the various disease groups may overlap to similar CERs, such as symptoms, office visits (or “encounters”), medications, or the like.

These summaries may be organized and presented to a user, such as a medical professional, medical insurance representative, medical laboratory technician, medical specialist, or the like, for assisting the user in understanding the medical condition of the patient in a concise and easy to access manner that does not require that the user sift through voluminous amounts of patient EMR data and make mental notes and correlations amongst the large amount of data. The particular summaries may be generated based on a specification of the types of medical episodes that the user wishes to be informed of. For example, the user may specify one or more characteristics of a type of medical episode that the user is interested in viewing summary information about, e.g., particular CERs of interest, e.g., specific diseases, medications, encounters, etc., severity of episodes, etc. Such an input from the user can be taken at set up time or each time a patient summary is desired through a user interface in which the user, e.g., a clinician, requests the type of information sought. Since the information recorded in the EMR for the patient may not be described in the same way as the request, a concept name mapping algorithm may be employed to translate such a request to a known vocabulary.

The summary may be output to the user in a graphical user interface with a drill down capability. That is, the user may select medical issue summaries or medical episode summaries and drill down into the particular sub-elements that contribute to the summary, e.g., the particular medical episodes that comprise the medical issue summary, the particular CERs associated with the selected medical episode, or the like. Thus, the user may be presented a graphical user interface with the summaries corresponding to the user's specified interest, and may then select the summaries within the graphical user interface where the user wishes to see more detailed information about the medical episode.

Hence, the mechanisms of the illustrative embodiments provide an improved computerized tool for analyzing voluminous patient medical information, such as in a consolidated patient electronic medical record (EMR) where data from a variety of different medical computing systems are compiled, and detecting medical episodes within this patient medical information. In some illustrative embodiments, the computerized tools further identify correlations amongst medical episodes that are indicative of a medical issue associated with the patient. This medical episode and medical issue information may be stored in association with the patient medical information or patient EMR data for use in outputting graphical user interfaces (GUIs) comprising summaries of medical episodes and/or medical issues for users. The particular summaries presented in the GUIs may be based on user specified criteria. Moreover, the summaries are selectable and able to be drilled down into obtain the details of the particular summary, e.g., CERs, individual episodes that make up a medical issue, etc. These mechanisms alleviate the burden on users to sift through large amounts of medical data to identify medical data relevant to the particular purpose for which the user wishes to access the patient medical information.

Before beginning the discussion of the various aspects of the illustrative embodiments in more detail, it should first be appreciated that throughout this description the term “mechanism” will be used to refer to elements of the present invention that perform various operations, functions, and the like. A “mechanism,” as the term is used herein, may be an implementation of the functions or aspects of the illustrative embodiments in the form of an apparatus, a procedure, or a computer program product. In the case of a procedure, the procedure is implemented by one or more devices, apparatus, computers, data processing systems, or the like. In the case of a computer program product, the logic represented by computer code or instructions embodied in or on the computer program product is executed by one or more hardware devices in order to implement the functionality or perform the operations associated with the specific “mechanism.” Thus, the mechanisms described herein may be implemented as specialized hardware, software executing on general purpose hardware, software instructions stored on a medium such that the instructions are readily executable by specialized or general purpose hardware, a procedure or method for executing the functions, or a combination of any of the above.

The present description and claims may make use of the terms “a”, “at least one of”, and “one or more of” with regard to particular features and elements of the illustrative embodiments. It should be appreciated that these terms and phrases are intended to state that there is at least one of the particular feature or element present in the particular illustrative embodiment, but that more than one can also be present. That is, these terms/phrases are not intended to limit the description or claims to a single feature/element being present or require that a plurality of such features/elements be present. To the contrary, these terms/phrases only require at least a single feature/element with the possibility of a plurality of such features/elements being within the scope of the description and claims.

Moreover, it should be appreciated that the use of the term “engine,” if used herein with regard to describing embodiments and features of the invention, is not intended to be limiting of any particular implementation for accomplishing and/or performing the actions, steps, processes, etc., attributable to and/or performed by the engine. An engine may be, but is not limited to, software, hardware and/or firmware or any combination thereof that performs the specified functions including, but not limited to, any use of a general and/or specialized processor in combination with appropriate software loaded or stored in a machine readable memory and executed by the processor. Further, any name associated with a particular engine is, unless otherwise specified, for purposes of convenience of reference and not intended to be limiting to a specific implementation. Additionally, any functionality attributed to an engine may be equally performed by multiple engines, incorporated into and/or combined with the functionality of another engine of the same or different type, or distributed across one or more engines of various configurations.

In addition, it should be appreciated that the following description uses a plurality of various examples for various elements of the illustrative embodiments to further illustrate example implementations of the illustrative embodiments and to aid in the understanding of the mechanisms of the illustrative embodiments. These examples intended to be non-limiting and are not exhaustive of the various possibilities for implementing the mechanisms of the illustrative embodiments. It will be apparent to those of ordinary skill in the art in view of the present description that there are many other alternative implementations for these various elements that may be utilized in addition to, or in replacement of, the examples provided herein without departing from the spirit and scope of the present invention.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

As noted above, the present invention provides mechanisms for generating a summary output of a patient's medical information specifically with regard to the automatic identification of medical episodes and/or medical issues comprising correlated medical episodes. The mechanisms of the illustrative embodiments process the patient medical information, which may be present for example in a patient's EMR compiled from a variety of different source computing systems, to extract comparative effective research (CER) elements. The processing of the patient medical information may comprise performing natural language processing of the patient medical information in the patient EMR to extract terms/phrases indicative of medical concepts, such as based on medical knowledge resources. The medical knowledge resources may comprise dictionary data structures for medical terminology, pharmaceutical reference texts and/or medication information resource data structures generated by pharmaceutical providers or medical organizations, medical journals, medical research documents, and the like, which may be processed during an ingestion process to extract medical concepts and corresponding terms/phrases, etc. Moreover, medical coding scheme documentation, laboratory notation resource documentation, and the like, may also be used to identify elements of patient medical information that are indicative of particular recognizable medical concepts. Any source of medical knowledge that may be represented in a data structure which can then be used to match to instances of the alphanumeric strings in patient medical information may be used without departing from the spirit and scope of the illustrative embodiments.

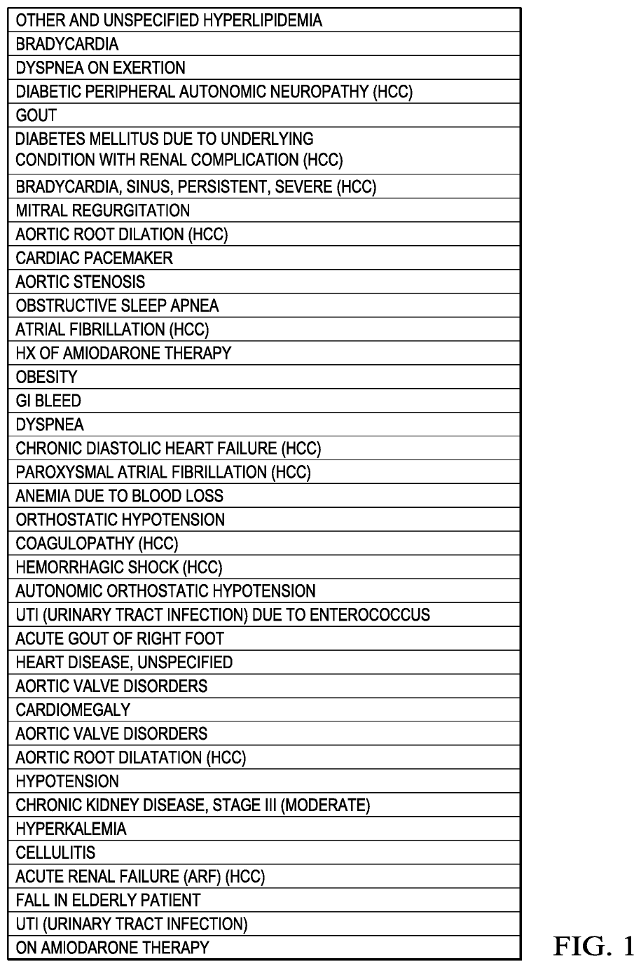

For example, processing the compiled patient EMR may result in instances of CERs corresponding to medical conditions, e.g., diseases, being extracted from the patient EMR based on the correlation of text/clinical values/medical codes in the patient EMR with instances in the medical resources utilized to extract the instances of CERs. These medical conditions may together constitute a “problem list” that is generated from the patient EMR indicating the various medical conditions or problems that the patient has experienced or which are currently associated with the patient. FIG. 1 is an example of a portion of CER instances extracted from patient information in a patient EMR which may constitute a problem list for the patient. The listing in FIG. 1 represents the actual names present in the problem list of a single patient record over a period of 5 years. When considering recorded or billable diagnosis, a typical patient's record may have more than 1000 diagnoses and corresponding CERs in the problem listing.

The extracted instances of the CERs from the patient EMR may have corresponding attributes associated with the instances including time stamps or other temporal attributes indicating a time when the corresponding instance of the CER was added to the patient medical information or a time at which the event or diagnosis occurred, the medication was prescribed or administered, the time at which the signs or symptoms were indicated to be present with the patient, or the like. While the instances of the CERs may have associated temporal attributes, and may have these temporal attributes plotted along a time series, it is still difficult to discern correlations between these CER instances when taking into consideration a large number of different types of CERs present in the patient EMR or patient medical information.

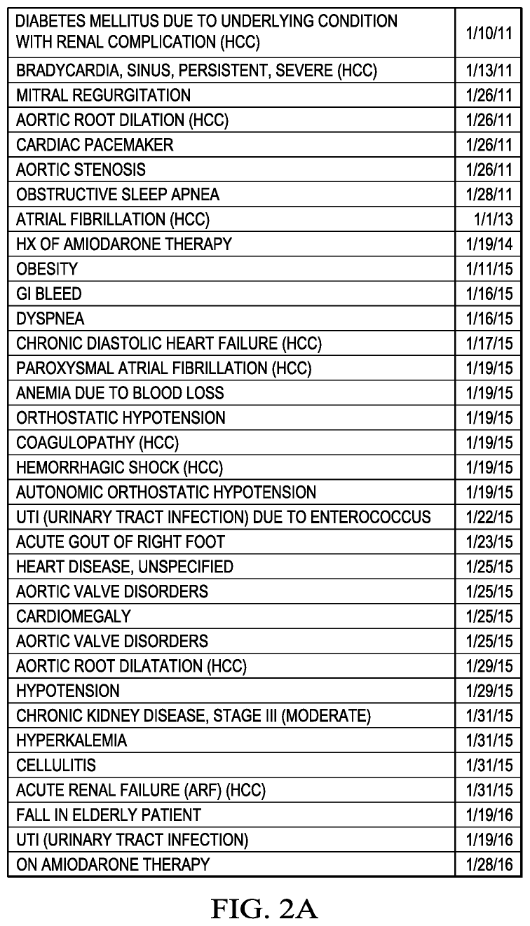

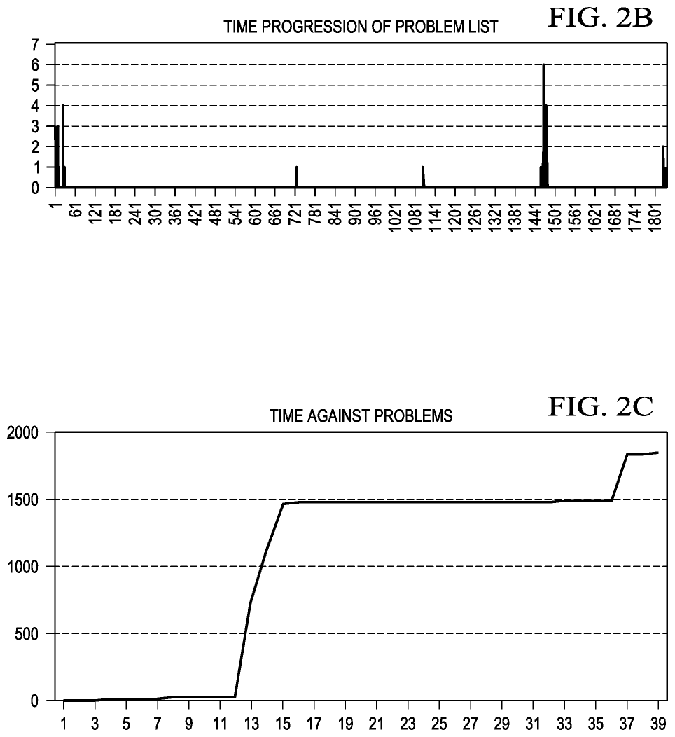

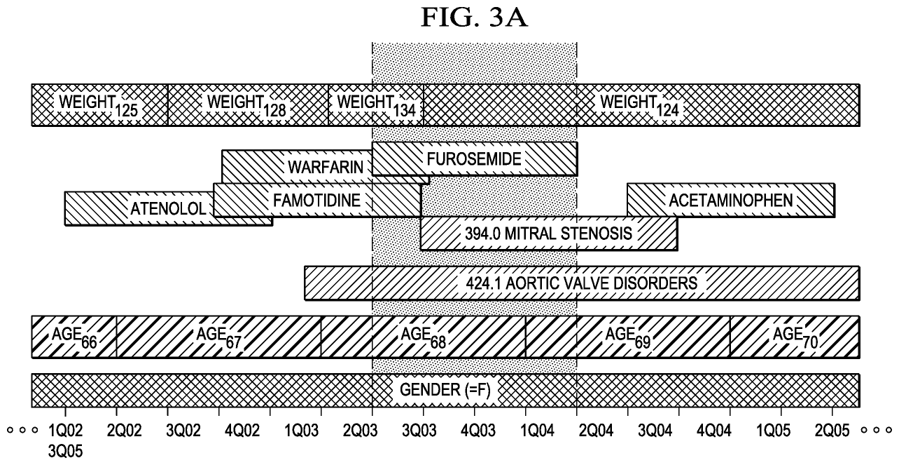

The automated medical episode identification and summarization engine of the illustrative embodiments provides a computerized tool that performs automatic detection and reporting of medical episodes in patient medical history based on the analysis of a time series of CER instances present in the patient EMR data, as may be generated based on the temporal attributes of the instances of CERs extracted from the patient EMR data. As noted above, these CERs are time varying and are mapped to a time series for each CER, such that each of the CERs may be plotted as a separate time series. FIGS. 2A-2C are example diagrams illustrating a time progression of a problem list of CER instances that may be generated based on temporal attributes of the CER instances. FIG. 2A illustrates the problem list that may be extracted from the patient medical information of the patient EMR along with timestamp information. FIG. 2B shows a timeline plot in which spikes in the timeline plot are indicative of an event used for forming episodes. Frequently these events are visits to encounter medical personnel, time points at which a medication is prescribed, when lab results are received, etc. FIG. 2C shows another plot of a problem list against time on a different time scale.

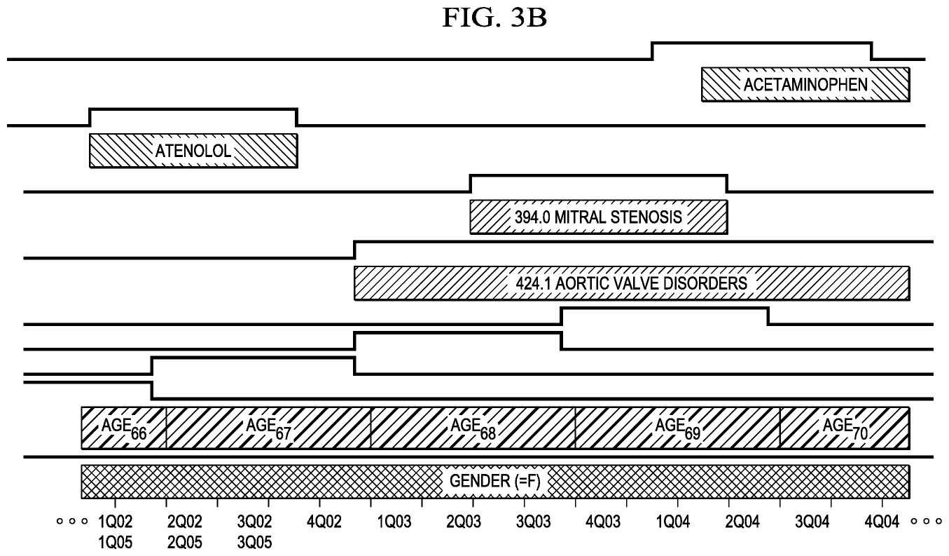

FIGS. 3A and 3B are example diagrams illustrating an extract of a patient record indicating the demographic information, the medications prescribed, and the diseases diagnosed for the patient. The co-occurrence of information can be seen against the patient timeline. These incidents are modeled as a unit time series in which the value is 1 for the period of time the event occurred and 0 at other times. By cumulating these scores registered in time (i.e. summing the 1s per time instant), a cumulative time series of the combined CER variables is formed which supports identifying the medical episodes.

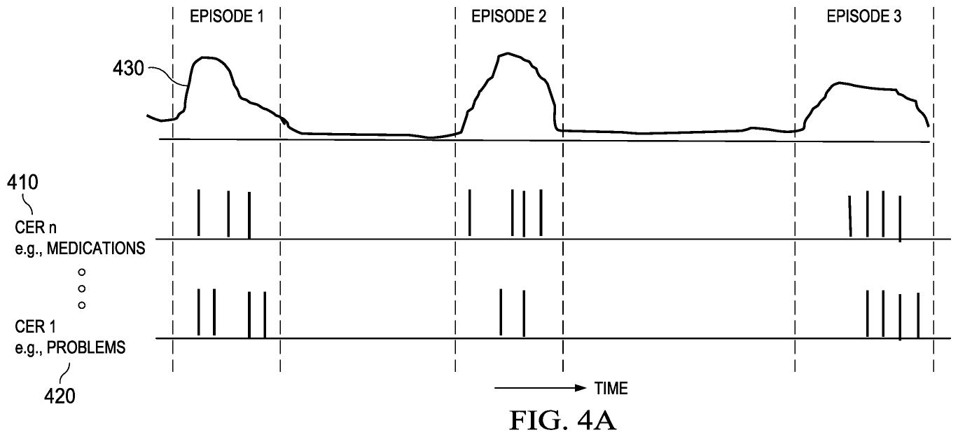

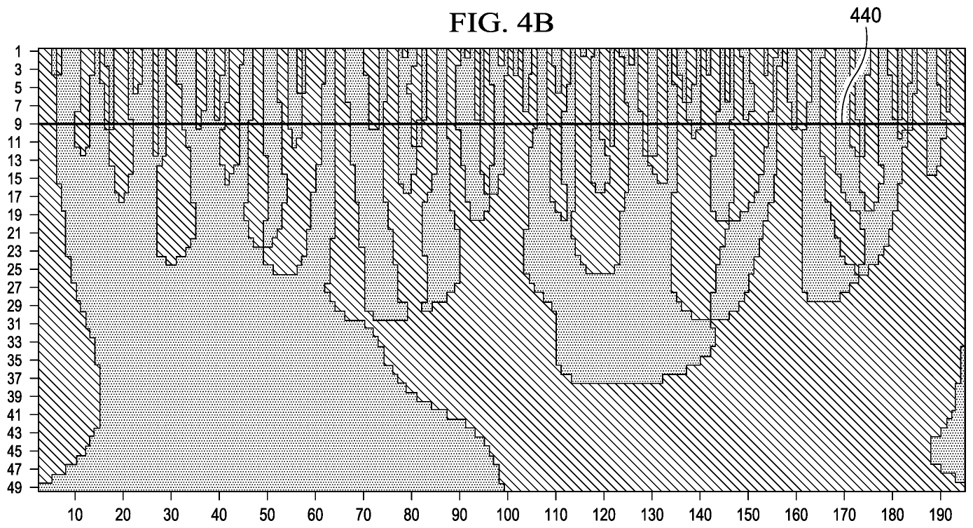

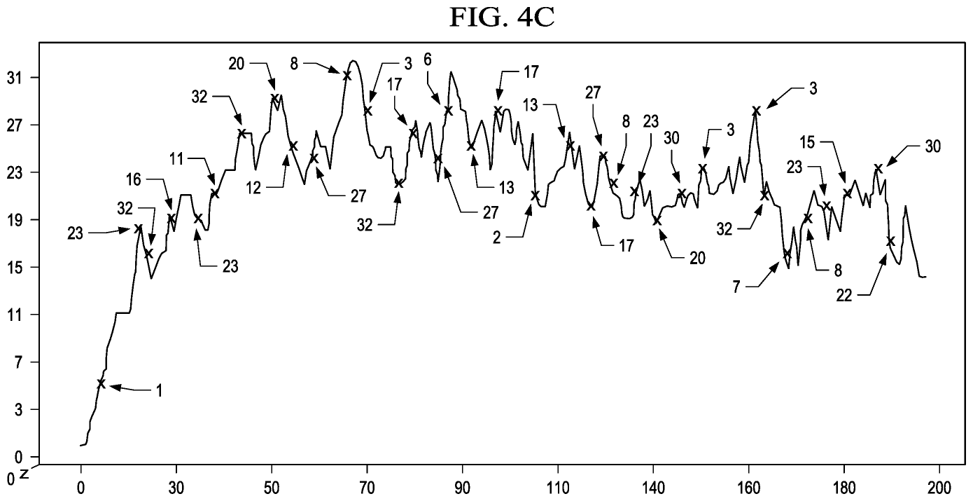

FIG. 4A is an example diagram illustrating a multi-dimensional CER time series cumulative profile in accordance with one illustrative embodiment. As shown in FIG. 4A, the individual CER time series 410-420 may be associated with different types of CERs with instances of the CERs present in the patient medical information causing spikes to occur in the time series indicating the presence of these CERs at particular time points. Combining these time series in a multi-dimensional CER time series and performing interpolation results in a curve 430 indicative of a medical episode signal.

A scale space analysis, e.g., Gaussian scale space analysis, is applied to the multi-dimensional time series to identify major inflection points in the multi-dimensional time series that identify the boundaries of medical episodes with the CERs between two inflection points being considered to be part of that medical episode. Scale-space theory is a framework for multi-scale signal representation developed by the computer vision, imaging processing and signal processing communities with complementary motivations from physics and biological vision. It is a formal theory for handling image structures at different scales by representing an image as a one-parameter family of smoothed images (i.e. the scale-space representation) parameterized by the size of the smoothing kernel used for suppressing fine-scale structures. The main type of scale space is the linear (Gaussian) scale space, which has wide applicability as well as the attractive property of being possible to derive from a small set of scale-space axioms. The corresponding scale-space framework encompasses a theory for Gaussian derivative operators, which can be used as a basis for expressing a large class of visual operations for computerized systems that process visual information. This framework also allows visual operations to be made scale invariant, which is necessary for dealing with the size variations that may occur in image data, because real-world objects may be of different sizes and in addition the distance between the object and the camera may be unknown and may vary depending on the circumstances.

While the general theory of scale space analysis is known in computer vision, the illustrative embodiments adapt scale space analysis for solving the problem of the detection of medical episodes from patient electronic medical records (EMRs). Specifically, the medical episodes can be defined as the interval between salient curvature change points in the cumulative time series of CER variables described earlier. Since such a time series can be modeled as a multi-dimensional curve, the illustrative embodiments can find salient curvature change points in the curve and use these to divide the time series into medical episodes. The salient curvature change points are those that are present even after smoothing the signal at multiple scales. By smoothing with a Gaussian kernel C̅(̅t̅,̅ σ̅)̅=C(t)*G(t, σ)=

$${\int }_{- ∞}^{∞} C ( u ) \frac{1}{σ \sqrt{2 π}} {e}^{- \frac{{( t - u )}^{2}}{2 {σ}^{2}}} ,$$

the result generated is a smoothed curve. The curvature changes can be recovered from the negative going zero crossings of the second derivative.

The curve as a function of scale appears as shown in FIG. 4B and the inflection points along with their rank in scale space are shown in FIG. 4C. The optimal scale is derived as minima of the combined objective function formed from the mean square error of interpolation using the samples at inflection points. The selected optimal scale is indicated by the line 440 in FIG. 4B. Once the scale is selected, the inflection points that exist at that scale are used to determine the episodic break points for the medical episodes. The interval between two consecutive episodic breakpoints that contain events, e.g., timeline instances of CERs, then becomes medical episode.

In some illustrative embodiments, medical episodes may be contiguous and non-overlapping based on this analysis. In other words, in some embodiments, the start and end boundaries for a medical episode are only applicable to a single episode, i.e. the same boundary does not constitute a boundary for multiple episodes, even if episodes overlap, such as shown in FIG. 4A where episodes 1, 2, and 3 have clearly different boundaries from one another. That is, a start boundary for a first episode at T1 cannot be a start boundary or end boundary for another second episode. This permits separate distinct medical episodes to be identified based on a pair of boundaries, e.g., starting at a first identified boundary, the next boundary is considered the end of the medical episode, with the next boundary in time order being the start of a next medical episode, and so on. In other illustrative embodiments, boundaries may be boundaries for multiple episodes and the same instances of CERs may exist within multiple episodes. Moreover, in some illustrative embodiments, medical episodes may overlap one another with some of the instances of CERs being in each of the overlapping episodes.

The medical episodes identified in this manner, by the mechanisms of the illustrative embodiments are further analyzed to identify commonalities and correspondences between the CER instances associated with the medical episodes. Patterns of types of instances of CERs associated with the same medical episode are cognitively evaluated to generate scores associated with different types of medical episodes. For example, various medications being administered, symptoms, office visits to the doctor, diagnoses, etc., may all be correlated to identify a medical episode type which may be used to summarize the medical episode.

The information about the type of medical episode and the data corresponding to instances of CERs associated with the medical episode are stored as a medical episode data structure associated with the patient EMR data. This may be performed for each medical episode identified via the above process by which a multi-dimensional time series of instances of CERs is generated, analyzed by scale-space analysis, and medical episode identification is determined based on the identified inflection points. The medical episode data structures may then be used to generate summaries of medical episodes associated with a patient's medical information.

For example, a patient may be diagnosed at an urgent care clinic in Florida, to have bronchitis, sinusitis, and an ear infection and may be prescribed antibiotics. The same patient may later have an encounter with their primary care physician in Texas where the physician diagnoses the patient with a chronic cough and congestion and provide the patient with a steroid injection and a steroid inhaler. The patient may then return to the physician at a later time complaining of shortness of breath, and the physician may diagnose the patient with bronchitis and prescribe another antibiotic. All of these events, based on the analysis performed by the mechanisms of the illustrative embodiments, may be identified as correlating to the same medical episode and may be summarized by the mechanisms of the illustrative embodiments, e.g., a respiratory viral infection medical episode. The medical episode data structure will store the details of the instances of CERs associated with the medical episode as extracted from the patient's medical information, as well as the results of the analysis performed by the mechanisms of the illustrative embodiments, such as the classification of the medical episode, duration of the medical episode as indicated by the boundaries, and the like. This medical episode data structure may then be used as the basis for generating a summary of the medical episode that may be presented to a user via a graphical user interface, for example, through which the user may drill-down into the details of the medical episode.

As mentioned previously, it should be appreciated that such summaries may be generated across medical episodes as well such that patterns of medical episodes may be identified which may then be correlated to a particular medical issue type, with a corresponding medical issue data structure being generated, which may then be used to summarize a plurality of medical episodes. For example, in some illustrative embodiments, groups of CERs that overlap may be generated in the manner described above, where the overlap is associated with common CERs associated with the different groups, e.g., the groups of CERs defining medical episodes may be centered around a particular CER and thus, the various disease groups may overlap to similar CERs.

The medical episode data structures and/or medical issue data structures, may be used as a basis for generating summaries of such medical episodes and medical issues. These summaries may be organized and presented to a user, e.g., medical professional, for assisting the user in understanding the medical condition of the patient in a concise and easy to access manner. The particular summaries may be generated based on a specification of the types of medical episodes that the user wishes to be informed of, e.g., the user may specify one or more CERs or other criteria of a type of medical episode that the user is interested in viewing summary information about. The resulting graphical user interface (GUI) generated with the various summaries may be output to the user via their client computing device. The summaries in the GUI may be user selectable so as to implement a drill down capability. In this way, the user may select medical issue summaries or medical episode summaries and drill down into the particular sub-elements that contribute to the summary. Thus, the user may be presented a graphical user interface with the summaries corresponding to the user's specified interest, and may then select the summaries within the graphical user interface where the user wishes to see more detailed information about the medical episode.

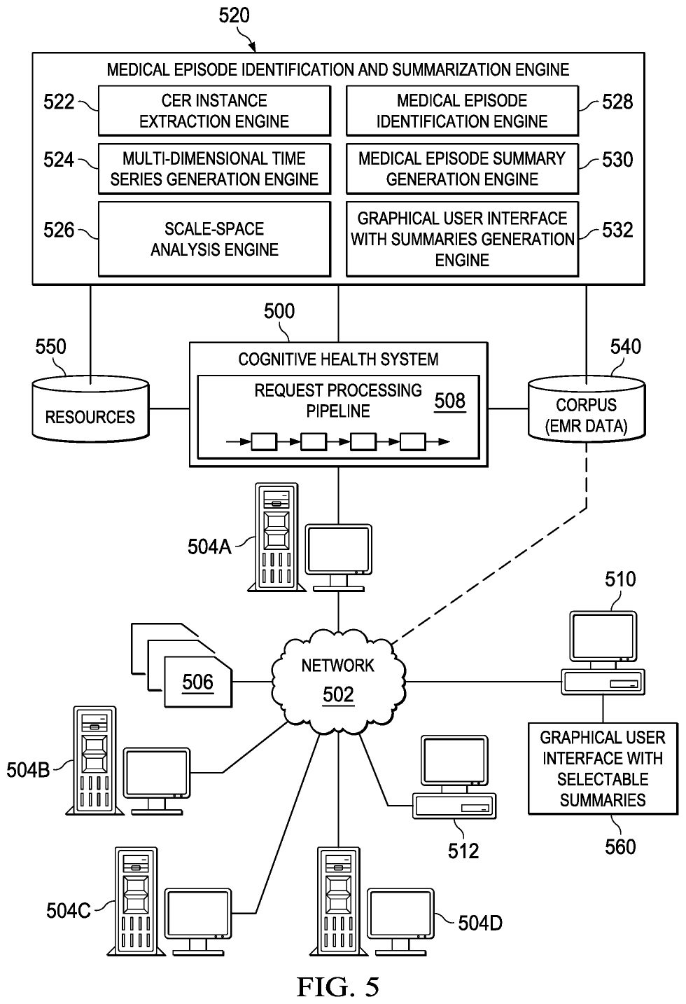

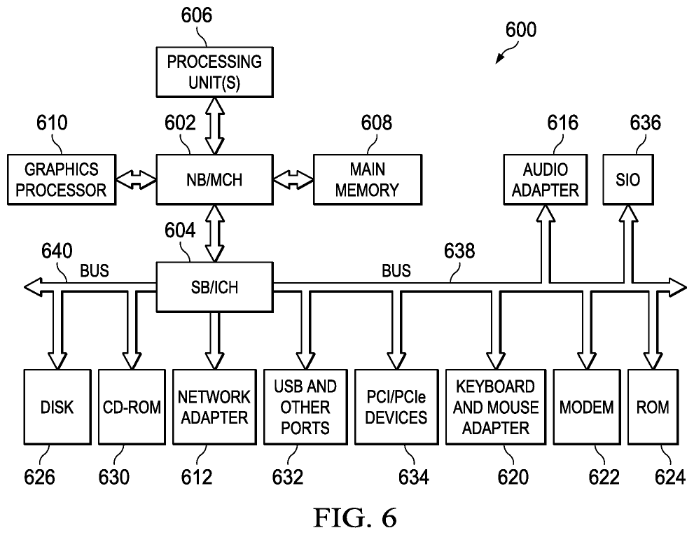

It is clear from the above, that the illustrative embodiments are rooted in computer technology and define a new type of computing tool for generating and outputting summaries of medical imaging studies. Thus, it is apparent to those of ordinary skill in the art that the mechanisms of the illustrative embodiments may be implemented in various types of data processing environments as a computing tool. FIGS. 5 and 6 are examples of computing or data processing environments in which aspects of the illustrative embodiments may be implemented. It should be appreciated that FIGS. 5 and 6 are only examples and are not intended to assert or imply any limitation with regard to the environments in which aspects or embodiments of the present invention may be implemented. Many modifications to the depicted environments may be made without departing from the spirit and scope of the present invention.

FIG. 5 depicts a schematic diagram of one illustrative embodiment of a cognitive health computing system 500 implementing a request processing pipeline 508 in a computer network 502. The cognitive health computing system 500 is implemented on one or more computing devices 504A-D (comprising one or more processors and one or more memories, and potentially any other computing device elements generally known in the art including buses, storage devices, communication interfaces, and the like) connected to the computer network 502. For purposes of illustration only, FIG. 5 depicts the cognitive health computing system 500 being implemented on computing device 504A only, but the cognitive health computing system 500 may be distributed across multiple computing devices, such as a plurality of computing devices 504A-D. The network 502 includes multiple computing devices 504A-D, which may operate as server computing devices, and 510-512 which may operate as client computing devices, in communication with each other and with other devices or components via one or more wired and/or wireless data communication links, where each communication link comprises one or more of wires, routers, switches, transmitters, receivers, or the like.

In some illustrative embodiments, the cognitive health computing system 500 and network 502 enables request processing functionality for one or more cognitive health computing system users via their respective computing devices 510-512. That is, a user may submit a request to the cognitive health computing system 500 via their client computing device 510, for example, to request viewing of a particular patient's electronic medical record or medical information, which may be stored in a corpus in a network storage system 506 and/or corpus storage 540 which may be directly attached to the cognitive health system 500 and/or network attached. The patient's electronic medical record (EMR) or medical information may be compiled from one or a plurality of source computing systems which may be associated with different medical service providers and/or other organizations associated with the medical field, for example, such as pharmacies, hospitals, doctor offices, medical laboratories, medical insurance providers, and the like.

The request submitted by the user, such a of client device 510, may specify the particular patient whose medical information or EMR is to be viewed and one or more criteria for specifying a type of patient medical information or EMR summary that is to be generated, e.g., the purpose for requesting access to the patient medical information or EMR. The medical image study viewing computing system 200 may then operate on the request by processing the specified medical imaging study in accordance with an illustrative embodiment as described herein, to generate the medical imaging study summary output having salient medical images selected from the medical imaging study provided in a storyboard representation of a graphical user interface that is provided back to the requestor via the client computing device 210.

The cognitive health computing system 500 may be configured to implement a request processing pipeline 508 that receives inputs from various sources. The requests may be posed in the form of a natural language question, natural language request for information, natural language request for the performance of a cognitive operation, or the like. For example, the cognitive health computing system 500 receives input from the network 502, a corpus or corpora of documents or data structures 506, 540, cognitive system users, and/or other data and other possible sources of input. In one embodiment, some or all of the inputs to the cognitive health computing system 500 are routed through the network 502. The various computing devices 504A-D on the network 502 include access points for content creators and cognitive system users.

In some illustrative embodiments, the computing devices 504A-D may include devices for a storing one or more databases representing the corpus or corpora of data 506, 540 (which are shown as a separate entities in FIG. 5 for illustrative purposes only). Portions of the corpus or corpora of data 506, 540 may also be provided on one or more other network attached storage devices, in one or more databases, or other computing devices not explicitly shown in FIG. 5. The network 502 includes local network connections and remote connections in various embodiments, such that the cognitive health computing system 500 may operate in environments of any size, including local and global, e.g., the Internet.

In one embodiment, the content creator computing systems may provide content for the corpora 506, 540 in a document or data structure of the corpus or corpora of data 506, 540 for use as part of a corpus of data with the cognitive health computing system 500. The document or data structure includes any file, text, article, or source of data for use in the cognitive health computing system 500. Cognitive health computing system users access the cognitive health computing system 500 via a network connection or an Internet connection to the network 502, and input requests to the cognitive health computing system 500 that are processed based on the content in the corpus or corpora of data 506, 540.

In one embodiment, the requests are formed using natural language. The cognitive health computing system 500 may comprise natural language processing and/or cognitive processing engines which parse and interpret the request, and perform the corresponding operations for generating a requested output, which includes the operations of the illustrative embodiments to generate a patient medical information or patient EMR summary output in which medical episodes are summarized, via the pipeline 508. The results are provided as a response to the cognitive health computing system user that is transmitted to their client computing device, e.g., client computing device 510. The pipeline 508 implemented by the cognitive health computing system 500 may comprise a plurality of stages for processing an input request based on information obtained from the corpus or corpora of data 506, 540.

In some illustrative embodiments, the cognitive health computing system 200 may implement at least a portion of the request processing pipeline 508 as a Question Answering (QA) pipeline (also referred to as a Question/Answer pipeline or Question and Answer pipeline). The request processing pipeline 508 may be augmented to include logic stages for processing of queries generated from an input question or request using the medical episode identification and summarization engine 520, described hereafter, which implements the processes previously described above for identifying medical episodes and summarizing them for presentation to a user. For example, a user may submit a question to the cognitive system asking to view a particular patient's EMR with specific emphasis on cardiac episodes, i.e. “What cardiac episodes has patient P encountered?” As part of answering this question, the cognitive system may access patient electronic medical records, resource data structures 550 that represent medical knowledge, dictionary data structures, medical coding schemes, medical concepts, and the like, to perform a natural language text based analysis of such data structures from the corpus 506, 540, and in addition invoke the mechanisms of the illustrative embodiments to generate a medical episode summary based GUI to be output to the requestor that summarizes the medical information or EMR of the patient with regard to various medical episodes and/or medical issues associated with the patient. The GUI may focus on medical episodes and medical issues that are specifically associated with the criteria specified by the requestor in the request that indicates the medical episodes and issues that are of particular interest to the requestor.

The requests input by users via their client devices 510-512 and network 502 to the cognitive health computing system 500 may be provided as structure or unstructured request messages, natural language questions, or any other suitable format for requesting an operation to be performed by the cognitive health computing system 500 and medical episode identification and summarization engine 520. In one illustrative embodiment, the particular healthcare application that is implemented in the cognitive healthcare computing system 500 is a healthcare application for medical episode identification and summarization for decision support for a medical professional in which salient medical episodes (or collections of medical episodes, referred to as medical issues) are identified from the large amount of medical information present indicating individual events or occurrences with regard to the treatment of medical conditions of the patient. The salient medical episodes are identified by generating a multi-dimensional time series of instances of comparative effective research (CERs) of the same and/or different types, performing scale-space analysis on the multi-dimensional time series to generate inflection points indicative of medical episode boundaries, and the identifying the medical episodes and summarizing the medical episodes based on analysis of correlations or patterns of CERs within the medical episode boundaries. The resulting medical episode information is stored as medical episode data structures in association with the patient's medical information and/or EMR for use in generating a GUI with medical episode summaries. Thus, in subsequent requests, only the newest medical information need be processed to identify new medical episodes while previously identified medical episodes matching criteria for a requestor's request may be retrieved based on the medical episode data structures already stored in association with the patient medical information or EMR.

The medical episode/medical issue summaries generated by the operation of the medical episode identification and summarization engine 520 operating as part of, or in conjunction with, the cognitive health system 500 are put together into a graphical user interface (GUI) output that may be presented as results to a requestor that submitted the original request, e.g., GUI 560 which is output on the client computing system 510 from which the requestor sent the original request. It should be appreciated that the summaries presented in the GUI 560 are user selectable via GUI elements such that the user of the client computing device 510 may “drill-down” into the detailed data that is the basis for the individual summaries in the GUI 560. Thus, for example, the summary of a medical episode may be drilled down into to identify one or more of the CER instances that are part of that medical episode. Such drill down capability may have many layers, e.g., the user may subsequently drill down into the CER instance to obtain information about the particular event associated with the CER instance. The summaries presented in the GUI may be organized according to a determined level of relevancy to the criteria specified in the original request such that the summaries determined to be most relevant will be represented in the GUI more prominently than other summaries, for example.

It should be appreciated that the healthcare cognitive system, while shown as having a single request processing pipeline in the examples herein, may in fact have multiple request processing pipelines. Each request processing pipeline may be separately trained and/or configured to process requests associated with different domains or be configured to perform the same or different analysis on input requests (or questions in implementations using a QA pipeline), depending on the desired implementation. For example, in some cases, a first request processing pipeline may be trained and/or configured to operate on input requests directed to processing the medical information or patient EMRs with regard to a first type of medical episodes, e.g., cardiac episodes, while another processing pipeline may be trained and/or configured to operate on input requests directed to processing the medical information or patient EMR with regard to a second type of medical episode, e.g., pulmonary episodes. As another example, a third pipeline may be trained and configured to identify medical issues comprising a plurality of medical episodes, which may typically occur over longer periods of time. The various pipelines may operate in parallel on the same medical information and/or patient EMR data and may generate different medical episode/medical issue summaries based on their corresponding operations, each of which may be presentable to a user as a separate medical episode/medical issue summary, or may be integrated into the same medical episode/medical issue summary, in the same or different graphical user interfaces.

Moreover, each request processing pipeline may have their own associated corpus or corpora 506, 540 that they ingest and may be specifically directed to the particular operations for which the pipeline is trained and configured. In some cases, the request processing pipelines may each operate on the same domain of input requests but may have different configurations, e.g., different annotators or differently trained annotators, such that different analysis and potential results are generated. The cognitive health system 500 may provide additional logic for routing input requests to the appropriate request processing pipeline, such as based on a determined domain of the input request, e.g., the purpose of the medical episode summarization, combining and evaluating final results generated by the processing performed by multiple request processing pipelines, and other control and interaction logic that facilitates the utilization of multiple request processing pipelines.

As noted above, one type of request processing pipeline with which the mechanisms of the illustrative embodiments may be utilized is a Question Answering (QA) pipeline. It should be appreciated that while some illustrative embodiments may be described in the context of the cognitive system implementing one or more QA pipelines that operate on an input question, the illustrative embodiments are not limited to such. Rather, the mechanisms of the illustrative embodiments may operate on requests that are not posed as “questions” but are formatted as requests for the cognitive system to perform cognitive operations on a specified set of input data using the associated corpus or corpora and the specific configuration information used to configure the cognitive system. For example, rather than asking a natural language question of “What cardiac episodes occurred with patient P?”, the cognitive system may instead receive a request of “Generate a cardiac episode summary for patient P,” or the like. It should be appreciated that the mechanisms of the QA system pipeline, such as pipeline 508 for example, may operate on requests in a similar manner to that of input natural language questions with minor modifications. In fact, in some cases, a request may be converted to a natural language question for processing by the QA system pipelines if desired for the particular implementation.

As an overview, a cognitive system is a specialized computer system, or set of computer systems, configured with hardware and/or software logic (in combination with hardware logic upon which the software executes) to emulate human cognitive functions. These cognitive systems apply human-like characteristics to conveying and manipulating ideas which, when combined with the inherent strengths of digital computing, can solve problems with high accuracy and resilience on a large scale. A cognitive system performs one or more computer-implemented cognitive operations that approximate a human thought process as well as enable people and machines to interact in a more natural manner so as to extend and magnify human expertise and cognition. A cognitive system comprises artificial intelligence logic, such as natural language processing (NLP) based logic, for example, and machine learning logic, which may be provided as specialized hardware, software executed on hardware, or any combination of specialized hardware and software executed on hardware. The logic of the cognitive system implements the cognitive operation(s), examples of which include, but are not limited to, question answering, identification of related concepts within different portions of content in a corpus, intelligent search algorithms, such as Internet web page searches, for example, medical diagnostic and treatment recommendations, and other types of recommendation generation, e.g., items of interest to a particular user, potential new contact recommendations, or the like.

IBM Watson™ is an example of one such cognitive system which can process human readable language and identify inferences between text passages with human-like high accuracy at speeds far faster than human beings and on a larger scale. In general, such cognitive systems are able to perform the following functions:
Navigate the complexities of human language and understanding
Ingest and process vast amounts of structured and unstructured data
Generate and evaluate hypothesis
Weigh and evaluate responses that are based only on relevant evidence
Provide situation-specific advice, insights, and guidance
Improve knowledge and learn with each iteration and interaction through machine learning processes
Enable decision making at the point of impact (contextual guidance)
Scale in proportion to the task
Extend and magnify human expertise and cognition
Identify resonating, human-like attributes and traits from natural language
Deduce various language specific or agnostic attributes from natural language
High degree of relevant recollection from data points (images, text, voice) (memorization and recall)
Predict and sense with situational awareness that mimic human cognition based on experiences
Answer questions based on natural language and specific evidence

In one aspect, cognitive systems provide mechanisms for answering questions posed to these cognitive systems using a Question Answering pipeline or system (QA system) and/or process requests which may or may not be posed as natural language questions. The QA pipeline or system is an artificial intelligence application executing on data processing hardware that answers questions pertaining to a given subject-matter domain presented in natural language. The QA pipeline receives inputs from various sources including input over a network, a corpus of electronic documents or other data, data from a content creator, information from one or more content users, and other such inputs from other possible sources of input. Data storage devices store the corpus of data. A content creator creates content in a document for use as part of a corpus of data with the QA pipeline. The document may include any file, text, article, medical imaging data, or other source of data for use in the QA system. For example, a QA pipeline accesses a body of knowledge about the domain, or subject matter area, e.g., financial domain, medical domain, legal domain, etc., where the body of knowledge (knowledgebase) can be organized in a variety of configurations, e.g., a structured repository of domain-specific information, such as ontologies, or unstructured data related to the domain, or a collection of natural language documents about the domain.

Content users input questions to cognitive system which implements the QA pipeline. The QA pipeline then answers the input questions using the content in the corpus of data by evaluating documents, sections of documents, portions of data in the corpus, or the like. When a process evaluates a given section of a document for semantic content, the process can use a variety of conventions to query such document from the QA pipeline, e.g., sending the query to the QA pipeline as a well-formed question which is then interpreted by the QA pipeline and a response is provided containing one or more answers to the question. Semantic content is content based on the relation between signifiers, such as words, phrases, signs, and symbols, and what they stand for, their denotation, or connotation. In other words, semantic content is content that interprets an expression, such as by using Natural Language Processing.

The QA pipeline receives an input question, parses the question to extract the major features of the question, uses the extracted features to formulate queries, and then applies those queries to the corpus of data. Based on the application of the queries to the corpus of data, the QA pipeline generates a set of hypotheses, or candidate answers to the input question, by looking across the corpus of data for portions of the corpus of data that have some potential for containing a valuable response to the input question. The QA pipeline then performs deep analysis on the language of the input question and the language used in each of the portions of the corpus of data found during the application of the queries using a variety of reasoning algorithms. There may be hundreds or even thousands of reasoning algorithms applied, each of which performs different analysis, e.g., comparisons, natural language analysis, lexical analysis, or the like, and generates a score. For example, some reasoning algorithms may look at the matching of terms and synonyms within the language of the input question and the found portions of the corpus of data. Other reasoning algorithms may look at temporal or spatial features in the language, while others may evaluate the source of the portion of the corpus of data and evaluate its veracity.

The scores obtained from the various reasoning algorithms indicate the extent to which the potential response is inferred by the input question based on the specific area of focus of that reasoning algorithm. Each resulting score is then weighted against a statistical model. The statistical model captures how well the reasoning algorithm performed at establishing the inference between two similar passages for a particular domain during the training period of the QA pipeline. The statistical model is used to summarize a level of confidence that the QA pipeline has regarding the evidence that the potential response, i.e. candidate answer, is inferred by the question. This process is repeated for each of the candidate answers until the QA pipeline identifies candidate answers that surface as being significantly stronger than others and thus, generates a final answer, or ranked set of answers, for the input question.

Thus, in some illustrative embodiments, the medical episode identification and summarization engine 520 may be a separate independent computing system which provides functionality for viewing medical information or patient EMRs and is augmented to include the mechanisms of the illustrative embodiments for generating medical episode/medical issue summaries based on the generation of a multi-dimensional time series of CER instances extracted from patient medical information or a patient EMR, analyzing the multi-dimensional time series using scale-space analysis to identify boundaries of medical episodes, and then analyzing the patterns of CER instances within a medical episode to summarize the medical episode, as well as in some illustrative embodiments performing such analysis over multiple episodes to identify medical issues associated with the patient. In other illustrative embodiments, the medical episode identification and summarization engine 520 may operate in conjunction with, or be integrated with, the cognitive health computing system 500 that is configured to provide healthcare cognitive operations, such as medical decision support functionality for medical personnel, such as doctors, nurses, technicians, and the like, to assist them in making determinations as to how to treat a patient. In these various illustrative embodiments, the computing systems are modified to include logic implemented in specialized hardware, specialized software executed on hardware, or any combination of specialized hardware and software executed on hardware, for implementing the medical episode identification and summarization engine 520.

As shown in FIG. 5, the medical episode identification and summarization engine 520 comprises a CER instance extraction engine 522, a multi-dimensional time series generation engine 524, a scale-space analysis engine 526, a medical episode identification engine 528, a medical episode summary generation engine 530, and a GUI with summaries generation engine 532. The medical episode identification and summarization engine 520 may also utilize the various medical knowledge resources 550 available to the cognitive health system 500 as well as access medical information or patient EMRs from the corpus 540 and/or network attached storage 506, as further sources of data used to perform the functions of the medical episode identification and summarization engine 520. It should be appreciated that, in some illustrative embodiments, the medical episode identification and summarization engine 520 may be implemented as an additional stage within the pipeline 508 or may be invoked by a stage of processing in the pipeline 508 as part of the processing of a request received from a requestor utilizing a computing device such as client computer 510, for example.

In response to invocation of the medical episode identification and summarization engine 520, such as in response to receiving a request from a client computing device, e.g., client computing device 510, to provide medical episode information regarding a specified patient, with optional specified criteria indicating the types of medical episodes of interest to the user, the medical episode identification and summarization engine 520 accesses the corresponding patient medical information or EMR from storage systems associated with the corpus or corpora, e.g., one or more storage systems storing corpora 506, 540. The medical information or EMR comprises a plurality of data entries which may originate from a variety of different source computing systems which may be associated with the same or different medical or health service provider organizations. These entries preferably specify different events occurring with regard to the patient and have corresponding temporal characteristics, e.g., timestamps, indicating the dates/times when such events occurred. These entries may be in natural language format, as numerical values such as in the case of laboratory results, may be in specified formats, may identify medical codes according to one or more medical coding schemes, and the like.

The natural language processing and other analytic logic of the cognitive health system 500 and pipeline 508 may be utilized to analyze such medical information and/or EMR data to extract key terms/phrases, medical codes, etc. which may be correlated with CERs of interest by the CER instance extraction engine 522, to thereby generate a collection of CER instances and their associated temporal characteristics. It should be appreciated that the CER instances may be of various types and may have various different time and CER value scales.

The various types of CER instances extracted from the patient medical information or EMR data are used to generate time series for the various CER types, which are then normalized so that the time series may be combined into a multi-dimensional time series. These operations are performed by the multi-dimensional time series generation engine 524 which generates the time series for the various CERs and analyzes the time series to determine an appropriate normalization for the time series and applies it to integrate the information for the various time series into a single multi-dimensional time series having a cumulative profile. The multi-dimensional time series generation engine 524 may perform interpolation between points along the multi-dimensional time series so as to generate a continuous curve time series to which a scale-space analysis may be applied.

The scale-space analysis engine 526 applies scale-space analysis to the multi-dimensional time series to determine salient change points along the multi-dimensional time series, e.g., inflection points indicative of the boundaries of medical episodes. The scale-space analysis engine 526 may utilize any known, or later developed, scale-space algorithm, such as a linear (Gaussian) scale-space algorithm or the like, to identify the inflection points indicative of the boundaries of medical episodes. That is, the scale-space algorithm determines the proper scale to apply to the multi-dimensional time series and then uses a negative peak detection algorithm to identify negative peaks (i.e. determining inflection points having a minimum value) of the CER instances in the curve of the multi-dimensional time series. These negative peaks indicate the boundaries of medical episodes.

The medical episode identification engine 528 processes the results of the scale-space analysis engine 526 to identify these negative peaks and determine pairings of these negative peaks indicative of separate medical episodes. In some illustrative embodiments, the medical episode identification engine 528 may further perform analysis to identify medical issues comprising multiple medical episodes of a similar type. For example, medical episodes associated with a same medical episode type over a longer period of time in the multi-dimensional time series may be considered indicative of a medical issue as opposed to an individual medical episode. Thus, the medical episode identification engine 528 may operate in conjunction with the medical episode summary generation engine 530 to identify similar medical episodes that likely are related to the same medical issue with the patient.

The medical episode summary generation engine 530 identifies the CER instances associated with each of the medical episodes identified by the medical episode identification engine 528 and identifies correlations and patterns of these CER instances. For example, in some illustrative embodiments, the medical episode summary generation engine 530 may apply cognitive evidence based evaluations of the CER instances to determine evidence ratings as to a classification of the medical episode into one of a plurality of different medical episode types. In other illustrative embodiments, a rules-based engine may be applied to the characteristics of the CER instances of a medical episode to determine which type of medical episode is most likely represented by the particular correlation or pattern of CER instances. The identified correlations and patterns indicate the type of the medical episode which may then be used to summarize the medical episode and generate a medical episode summary data structure that is stored in association with the medical information or patient EMR data in the corpus 506, 540.

The graphical user interface with summaries generation engine 532 uses the identified medical episodes and their corresponding summary data structures to generate a GUI with summaries that are relevant to the original request submitted by the requestor. That is, not all medical episodes associated with the patient may be of particular interest to the requestor, or some may be of relatively more importance than others. Thus, the GUI with summaries generation engine 532 may comprise logic for filtering the medical episodes and/or medical issues identified by the engine 520 so as to utilize only those of particular interest to the requestor, as indicated by the criteria in the request, when generating the GUI output that is returned to the requestor.

The summaries that are presented in the output to the requestor may comprise user selectable elements to allow the user to select summaries which are of particular interest to the user and initiate a drill-down functionality of the GUI to obtain more detailed information about the medical episode and/or medical issue that is summarized. For example, the user may utilize their input devices associated with the client computer 510 to interact with the GUI 560 that is presented in response to the user's original request, which includes the summaries pertinent to the request as generated by the medical episode identification and summarization engine 520. In response to the user selecting a summary present in the GUI, the GUI presents additional detailed information of the medical episodes that make up the medical issue (if the summary is a summary of a medical issue), the detailed information about the CER instances that make up the medical episode, and the like. As noted above, multiple levels of drill-down are made possible such that more and more detailed information may be presented.

Thus, through this process of processing medical information and/or patient EMR data, large complex collections of medical information may be summarized based on the automated identification of medical episodes within the voluminous amount of medical information associated with the patient. The illustrative embodiments provide mechanisms for identifying which events, medical concepts, or CER instances, are associated with different medical episodes using multi-dimensional time series generation and scale-space analysis to identify the boundaries of these medical episodes and the CER instances that are associated with these medical episodes. The illustrative embodiments provide mechanisms for analyzing patterns or correlations between CER instances of a medical episode such that the medical episode may be summarized for presentation to a user as part of a GUI with medical episode summaries. This all leads to a more user accessible understanding of a patient's medical information to assist the user in understanding the medical condition of the patient and provide support for medical decision making.

As is evident from the above, the mechanisms of the illustrative embodiments are rooted in the computer technology arts and are implemented using logic present in such computing or data processing systems. These computing or data processing systems are specifically configured, either through hardware, software, or a combination of hardware and software, to implement the various operations described above. Thus, having been configured to perform these specific operations, the resulting configured computing or data processing systems are not generic computing or data processing systems simply performing generic computing operations or functions. To the contrary, the specifically configured computing devices or data processing systems are specific non-generic computing devices or data processing systems. As such, FIG. 6 is provided as an example of one type of data processing system in which aspects of the present invention may be implemented through specific configuration of the data processing system via the loading of software into memory and execution of that particular software on one or more processors of the data processing system to perform the described operations. Many other types of data processing systems may be likewise configured to specifically implement the mechanisms of the illustrative embodiments.

FIG. 6 is a block diagram of an example data processing system in which aspects of the illustrative embodiments are implemented. Data processing system 600 is an example of a computer, such as server 504A or client 510 in FIG. 5, for example, in which computer usable code or instructions implementing the processes for illustrative embodiments of the present invention are located. In one illustrative embodiment, FIG. 6 represents a server computing device, such as a server 504A, which implements a cognitive health computing system 500 augmented to include the additional mechanisms of the medical episode identification and summarization engine 520 in accordance with one or more of the illustrative embodiments described above with regard.

In the depicted example, data processing system 600 employs a hub architecture including North Bridge and Memory Controller Hub (NB/MCH) 602 and South Bridge and Input/Output (I/O) Controller Hub (SB/ICH) 604. Processing unit 606, main memory 608, and graphics processor 610 are connected to NB/MCH 602. Graphics processor 610 is connected to NB/MCH 602 through an accelerated graphics port (AGP).

In the depicted example, local area network (LAN) adapter 612 connects to SB/ICH 604. Audio adapter 616, keyboard and mouse adapter 620, modem 622, read only memory (ROM) 624, hard disk drive (HDD) 626, CD-ROM drive 630, universal serial bus (USB) ports and other communication ports 632, and PCI/PCIe devices 634 connect to SB/ICH 604 through bus 638 and bus 640. PCI/PCIe devices may include, for example, Ethernet adapters, add-in cards, and PC cards for notebook computers. PCI uses a card bus controller, while PCIe does not. ROM 624 may be, for example, a flash basic input/output system (BIOS).

HDD 626 and CD-ROM drive 630 connect to SB/ICH 604 through bus 640. HDD 626 and CD-ROM drive 630 may use, for example, an integrated drive electronics (IDE) or serial advanced technology attachment (SATA) interface. Super I/O (SIO) device 636 is connected to SB/ICH 604.

An operating system runs on processing unit 606. The operating system coordinates and provides control of various components within the data processing system 600 in FIG. 6. As a client, the operating system is a commercially available operating system such as Microsoft® Windows 10®. An object-oriented programming system, such as the Java™ programming system, may run in conjunction with the operating system and provides calls to the operating system from Java™ programs or applications executing on data processing system 600.

As a server, data processing system 600 may be, for example, an IBM® eServer™ System p® computer system, running the Advanced Interactive Executive) (AIX®) operating system or the LINUX® operating system. Data processing system 600 may be a symmetric multiprocessor (SMP) system including a plurality of processors in processing unit 606. Alternatively, a single processor system may be employed.

Instructions for the operating system, the object-oriented programming system, and applications or programs are located on storage devices, such as HDD 626, and are loaded into main memory 608 for execution by processing unit 606. The processes for illustrative embodiments of the present invention are performed by processing unit 606 using computer usable program code, which is located in a memory such as, for example, main memory 608, ROM 624, or in one or more peripheral devices 626 and 630, for example.

A bus system, such as bus 638 or bus 640 as shown in FIG. 6, is comprised of one or more buses. Of course, the bus system may be implemented using any type of communication fabric or architecture that provides for a transfer of data between different components or devices attached to the fabric or architecture. A communication unit, such as modem 622 or network adapter 612 of FIG. 6, includes one or more devices used to transmit and receive data. A memory may be, for example, main memory 608, ROM 624, or a cache such as found in NB/MCH 602 in FIG. 6.

Those of ordinary skill in the art will appreciate that the hardware depicted in FIGS. 5 and 6 may vary depending on the implementation. Other internal hardware or peripheral devices, such as flash memory, equivalent non-volatile memory, or optical disk drives and the like, may be used in addition to or in place of the hardware depicted in FIGS. 5 and 6. Also, the processes of the illustrative embodiments may be applied to a multiprocessor data processing system, other than the SMP system mentioned previously, without departing from the spirit and scope of the present invention.

Moreover, the data processing system 600 may take the form of any of a number of different data processing systems including client computing devices, server computing devices, a tablet computer, laptop computer, telephone or other communication device, a personal digital assistant (PDA), or the like. In some illustrative examples, data processing system 600 may be a portable computing device that is configured with flash memory to provide non-volatile memory for storing operating system files and/or user-generated data, for example. Essentially, data processing system 600 may be any known or later developed data processing system without architectural limitation.

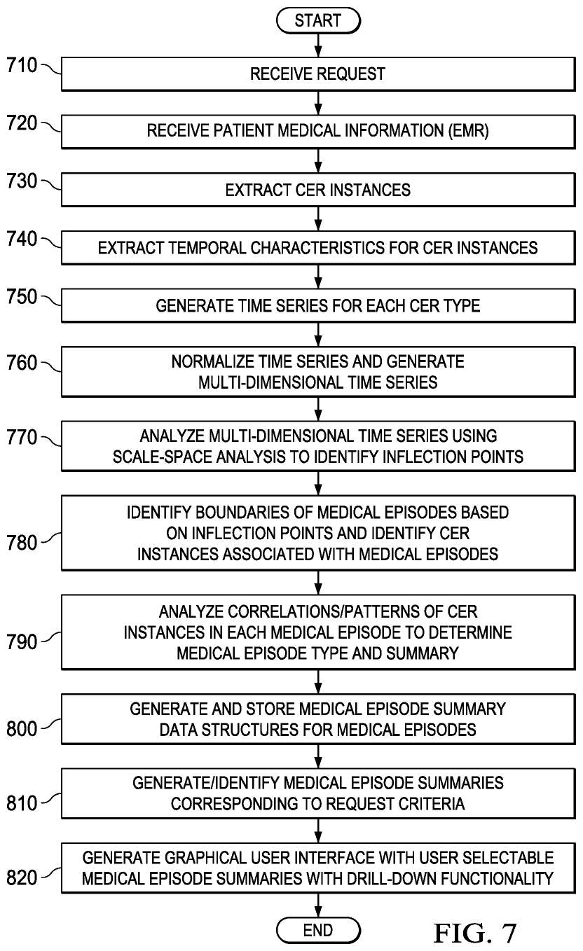

FIG. 7 is a flowchart outlining an example operation for identifying medical episodes in patient medical information and generating medical episode summaries, in accordance with one illustrative embodiment. As shown in FIG. 7, the operation starts by receiving a request to access patient medical information for a specific patient, where the request may specify criteria defining the medical episodes or issues of interest to the requestor (step 710). The request is processed and a lookup is performed in a corpus of medical information to identify the particular medical information or patient EMR data corresponding to the identified patient (step 720).

The medical information or patient EMR data is analyzed to extract CER instances based on medical knowledge resources, medical dictionaries, medical concept ontologies, medical coding scheme resource data, and/or any other source of medical knowledge that is indicative of medical concept instances, i.e. CER instances (step 730). This analysis may utilize various types of processing including processing specified formats of data, performing natural language processing, and/or the like. Temporal characteristics of the CER instances are also extracted in association with the CER instances (step 740) and, for each type of CER, a time series is generated (step 750).

The time series for the various types of CERs are normalized and integrated to generate a multi-dimensional time series for the collection of extracted CER instances (step 760). The multi-dimensional time series is analyzed utilizing a scale-space analysis algorithm to identify inflection points in the multi-dimensional time series (step 770). The inflection points are utilized to identify the boundaries of medical episodes and the corresponding CER instances associated with each of the medical episodes (step 780). The correlations and/or patterns of CER instances associated with a medical episode are analyzed to determine a type of the medical episode and a summary of the medical episode (step 790). A corresponding medical episode summary data structure is generated and stored in association with the patient medical information or patient EMR data (step 800).

The medical episodes whose types match a type corresponding to the criteria specified in the original request are identified and their corresponding summaries retrieved/generated (step 810). The summaries are utilized to generate a GUI representation of the patient medical information or patient EMR data in which the summaries are user selectable in order to invoke a drill-down functionality (step 820). That is, in response to a user selecting a summary, the GUI presents additional detailed information about the elements that make up that summary. The operation then terminates.

As noted above, it should be appreciated that the illustrative embodiments may take the form of an entirely hardware embodiment, an entirely software embodiment or an embodiment containing both hardware and software elements. In one example embodiment, the mechanisms of the illustrative embodiments are implemented in software or program code, which includes but is not limited to firmware, resident software, microcode, etc.

A data processing system suitable for storing and/or executing program code will include at least one processor coupled directly or indirectly to memory elements through a communication bus, such as a system bus, for example. The memory elements can include local memory employed during actual execution of the program code, bulk storage, and cache memories which provide temporary storage of at least some program code in order to reduce the number of times code must be retrieved from bulk storage during execution. The memory may be of various types including, but not limited to, ROM, PROM, EPROM, EEPROM, DRAM, SRAM, Flash memory, solid state memory, and the like.

Input/output or I/O devices (including but not limited to keyboards, displays, pointing devices, etc.) can be coupled to the system either directly or through intervening wired or wireless I/O interfaces and/or controllers, or the like. I/O devices may take many different forms other than conventional keyboards, displays, pointing devices, and the like, such as for example communication devices coupled through wired or wireless connections including, but not limited to, smart phones, tablet computers, touch screen devices, voice recognition devices, and the like. Any known or later developed I/O device is intended to be within the scope of the illustrative embodiments.

Network adapters may also be coupled to the system to enable the data processing system to become coupled to other data processing systems or remote printers or storage devices through intervening private or public networks. Modems, cable modems and Ethernet cards are just a few of the currently available types of network adapters for wired communications. Wireless communication based network adapters may also be utilized including, but not limited to, 802.11 a/b/g/n wireless communication adapters, Bluetooth wireless adapters, and the like. Any known or later developed network adapters are intended to be within the spirit and scope of the present invention.

The description of the present invention has been presented for purposes of illustration and description, and is not intended to be exhaustive or limited to the invention in the form disclosed. Many modifications and variations will be apparent to those of ordinary skill in the art without departing from the scope and spirit of the described embodiments. The embodiment was chosen and described in order to best explain the principles of the invention, the practical application, and to enable others of ordinary skill in the art to understand the invention for various embodiments with various modifications as are suited to the particular use contemplated. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method, in a data processing system comprising a processor and a memory, the memory comprising instructions executed by the processor to configure the processor to implement a medical episode analysis engine, the method comprising:
analyzing, by the medical episode analysis engine, patient information to extract a plurality of medical concept instances corresponding to medical characteristics associated with a patient;
generating, by the medical episode analysis engine, a multi-dimensional time series data structure representing a clinical history timeline of the medical concept instances;
analyzing, by the medical episode analysis engine, the multi-dimensional time series data structure to identify one or more medical episodes in the clinical history timeline, wherein a medical episode is a group of related encounters between one or more medical services providers and the patient; and
outputting, by the medical episode analysis engine, a visual representation of the identified one or more medical episodes to a computing device, wherein the visual representation provides a navigation user interface through which a user may browse the clinical history timeline associated with the patient.

2. The method of claim 1, wherein analyzing the multi-dimensional time series data structure comprises applying scale-space analysis, using an automatically determined scale, to analyze a multi-dimensional curve corresponding to the multi-dimensional time series data structure to identify inflection points indicative of the one or more medical episodes.

3. The method of claim 2, wherein medical concept instances present on the clinical history timeline between two inflection points in the multi-dimensional time series data structure correspond to a same medical episode in the one or more medical episodes.

4. The method of claim 1, wherein the one or more medical episodes comprise a plurality of medical episodes and wherein the method further comprises:
analyzing correlations between medical episodes in the plurality of medical episodes; and
grouping medical episodes together based on results of analyzing correlations between the medical episodes in the plurality of medical episodes.

5. The method of claim 4, wherein analyzing correlations between medical episodes in the plurality of medical episodes comprises applying medical knowledge resources, ontologies, or logic that identifies comparative effective research elements present within the medical episodes that are related with one another to thereby identify a higher level medical episode than the individual medical episodes that are grouped together.

6. The method of claim 4, wherein analyzing correlations between medical episodes in the plurality of medical episodes comprises identifying comparative effective research (CER) elements that are present in the medical episodes and identifying CER elements that are common to multiple medical episodes, and wherein medical episodes having common CER elements are grouped together.

7. The method of claim 1, wherein the navigation user interface provides an interface through which a user specifies a type of medical episode of interest, and wherein the method further comprises:
receiving a user input, via the navigation user interface, indicating one or more characteristics of one or more medical episodes of interest to the user;
searching the one or more medical episodes in the clinical history timeline based on the one or more characteristics to identify at least one matching medical episode; and
outputting, as part of the visual representation, the at least one matching medical episode.

8. The method of claim 7, wherein the one or more characteristics comprise a particular comparative effective research element of interest.

9. The method of claim 1, wherein the navigation user interface provides a user interface element for drilling down into a representation of a medical episode in the visual representation, to display details of comparative effective research elements present in the medical episode.

10. The method of claim 1, wherein the patient information comprises patient electronic medical records obtained from a plurality of different source computing systems associated with different medical service providers, and wherein the medical concept instances are pre-defined comparative effective research elements that are common to the plurality of different source computing systems.

11. A computer program product comprising a computer readable storage medium having a computer readable program stored therein, wherein the computer readable program, when executed on a computing device, causes the computing device to implement a medical episode analysis engine that operates to:
analyze patient information to extract a plurality of medical concept instances corresponding to medical characteristics associated with a patient;
generate a multi-dimensional time series data structure representing a clinical history timeline of the medical concept instances;
analyze the multi-dimensional time series data structure to identify one or more medical episodes in the clinical history timeline, wherein a medical episode is a group of related encounters between one or more medical services providers and the patient; and
output a visual representation of the identified one or more medical episodes to a computing device, wherein the visual representation provides a navigation user interface through which a user may browse the clinical history timeline associated with the patient.

12. The computer program product of claim 11, wherein the computer readable program causes the medical episode analysis engine to analyze the multi-dimensional time series data structure at least by applying scale-space analysis, using an automatically determined scale, to analyze a multi-dimensional curve corresponding to the multi-dimensional time series data structure to identify inflection points indicative of the one or more medical episodes.

13. The computer program product of claim 12, wherein medical concept instances present on the clinical history timeline between two inflection points in the multi-dimensional time series data structure correspond to a same medical episode in the one or more medical episodes.

14. The computer program product of claim 11, wherein the one or more medical episodes comprise a plurality of medical episodes and wherein the computer readable program further causes the medical episode analysis engine to:
analyze correlations between medical episodes in the plurality of medical episodes; and
group medical episodes together based on results of analyzing correlations between the medical episodes in the plurality of medical episodes.

15. The computer program product of claim 14, wherein the computer readable program causes the medical episode analysis engine to analyze correlations between medical episodes in the plurality of medical episodes at least by applying medical knowledge resources, ontologies, or logic that identifies comparative effective research elements present within the medical episodes that are related with one another to thereby identify a higher level medical episode than the individual medical episodes that are grouped together.

16. The computer program product of claim 14, wherein the computer readable program causes the medical episode analysis engine to analyze correlations between medical episodes in the plurality of medical episodes at least by identifying comparative effective research (CER) elements that are present in the medical episodes and identifying CER elements that are common to multiple medical episodes, and wherein medical episodes having common CER elements are grouped together.

17. The computer program product of claim 11, wherein the navigation user interface provides an interface through which a user specifies a type of medical episode of interest, and wherein the computer readable program further causes the medical episode analysis engine to:
receive a user input, via the navigation user interface, indicating one or more characteristics of one or more medical episodes of interest to the user;
search the one or more medical episodes in the clinical history timeline based on the one or more characteristics to identify at least one matching medical episode; and
output, as part of the visual representation, the at least one matching medical episode.

18. The computer program product of claim 17, wherein the one or more characteristics comprise a particular comparative effective research element of interest.

19. The computer program product of claim 11, wherein the navigation user interface provides a user interface element for drilling down into a representation of a medical episode in the visual representation, to display details of comparative effective research elements present in the medical episode.

20. An apparatus comprising:
a processor; and
a memory coupled to the processor, wherein the memory comprises instructions which, when executed by the processor, cause the processor to implement a medical episode analysis engine that operates to:
analyze patient information to extract a plurality of medical concept instances corresponding to medical characteristics associated with a patient; generate a multi-dimensional time series data structure representing a clinical history timeline of the medical concept instances;
analyze the multi-dimensional time series data structure to identify one or more medical episodes in the clinical history timeline, wherein a medical episode is a group of related encounters between one or more medical services providers and the patient; and
output a visual representation of the identified one or more medical episodes to a computing device, wherein the visual representation provides a navigation user interface through which a user may browse the clinical history timeline associated with the patient.
